(19) United States
(12) Patent Application Publication
(10) Pub. No.: US 2010/0098582 A1
(43) Pub. Date: Apr. 22, 2010

(54) Footwear decontaminating device and method

(76) Inventors: Devon C. Hansen, West Jordan, UT (US); Michael L. Rowzee, West Jordan, UT (US)

Correspondence Address: Michael L. Rowzee, 3431 Fenchurch Rd., West Jordan, UT, 84084, US

(21) Appl. No.: 12/288,132

(22) Filed: Oct. 16, 2008

(51) Int. Cl. A61L 2/18; A61L 2/26; B08B 1/00

(52) U.S. Cl. 422/30; 422/28; 422/37; 15/104.92

(57) ABSTRACT

A decontaminating device and method that decontaminates residue on and from footwear and other surfaces, configured as two basins with a decontaminating formulation in one basin and a deactivating formulation in the other basin. Footwear may be decontaminated while being worn, and this invention may be used to decontaminate for chemical residues, radiological residues, body fluids, and microorganisms.

### CROSS-REFERENCE TO RELATED APPLICATIONS

U.S. Pat. No. 2,989,965; Rod; Jun. 27, 1961
U.S. Pat. No. 3,696,459; Kucera, et al; Oct. 10, 1972
U.S. Pat. No. 5,071,628; Alazet; Dec. 10, 1991
U.S. Pat. No. 5,164,164; Strickler, et al; Nov. 17, 1992
U.S. Pat. No. 5,297,309; Rotoli; Mar. 29, 1994
U.S. Pat. No. 5,612,305; Lewis; Mar. 18, 1997
U.S. Pat. No. 5,881,427; Offner; Mar. 16, 1999
U.S. Pat. No. 6,146,588; Deighton; Nov. 14, 2000
U.S. Pat. No. 6,886,210; Dean; May 3, 2005
U.S. Pat. No. 6,925,896; Morton; Aug. 9, 2005
U.S. Pat. No. 7,299,811; Stewart, et al.; Nov. 27, 2007
International Patent KR100830552B; YOON; May 5, 2008
Primary Examiner: Michael L. Rowzee; Assistant Examiner: N/A; Attorney/Agent: N/A

### OTHER REFERENCE MATERIAL

Hazardous Chemical Spill Cleanup, Noyes Data Corp., 1979
National Pesticide Applicator Certification Core Manual, NASDA, No publication date provided
U.S. Army Field Manual 3-5, NBC Decontamination, 28 Jul. 2000
U.S. Army Field Manual 3-11, Multiservice Tactics, Techniques, and Procedures for Nuclear, Biological, and Chemical Defense Operations, Jan. 12, 2006

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC

Not Applicable

### BACKGROUND OF THE INVENTION

### Examples of Existing Art

### Footwear Cleaners and Sanitizers

A number of footwear cleaning and sanitizing devices have been invented. U.S. Pat. No. 2,989,965 describes a footwear decontaminating apparatus for use in the atomic industry. The user steps on a platform immersed in a fluid, and a high frequency agitator washes off the contaminating material. U.S. Pat. No. 3,696,459 is a floor mat assembly for cleansing and sanitizing ones shoes. A person steps into the first area where a liquid cleaning, and if wanted, sanitizing formulation cleans their shoes. The person steps into a second area and wipes his feet on an absorbent fabric to remove remaining soil and moisture. One of the patented footwear cleaning/disinfecting devices offers a rinse basin after the disinfecting basin. The rinse is with clear water.

A unique variation is a sticky device (sticky mat) to remove hair or other matter that may be used as evidence. The device (U.S. Pat. No. 6,925,896) is designed for use by police officials as they leave a crime scene. Not all devices in the present art are for people. Foot cleaning devices can be for dogs that live indoors and for livestock such as cows. A device for dog paws (U.S. Pat. No. 5,164,164) is designed to prevent the spread of specific organisms such as parvo, and non-specific organisms such as bacteria and mold. A livestock (cow) device (U.S. Patent Application 20040216692) is principally the same as the dog paw device except instead of parvo, it targets hoof and mouth disease.

### EMS/Firefighters/Police

Emergency medical personnel, firefighters, and police are often exposed to chemical hazards and pathogenic microorganisms at accident and crime scenes. Toxic chemicals may be on floors in commercial buildings where chemicals are used or stored, or in dwellings such as clandestine meth labs. Emergency personnel responding to the scene of an accident or crime often walk through body fluids (i.e. blood) that contains bloodborne pathogens such as Hepatitis “B” and human immunodeficiency virus (HIV).

Emergency personnel need to walk in and out of contaminated areas to retrieve items from vehicles, communicate with other officials, and collect evidence. Hazardous materials can be moved from indoors to public areas such as sidewalks and streets, and moved to the floors of emergency vehicles. When responding to major chemical incidents, emergency personnel usually operate a decontamination station for all exposed persons. At minor incidents, particularly where fire department personnel are not involved, no decontamination measures are taken except that personnel may scrape their feet as they walk to their vehicles.

### Military

Military personnel may be exposed to toxic chemicals and pathogenic organisms in battlefield environments. There are three levels of decontamination: immediate, operational, and thorough. Immediate decontamination is usually accomplished by self-decontamination or buddy-decontamination with available materials. Self/buddy measures are followed by operational or thorough decontamination. Operational decontamination consists of decontaminating personnel, vehicles, and equipment sufficiently to allow continuation of an ongoing mission. Ultimately, soldiers will be thoroughly decontaminated at a decontamination station.

In situations where soldiers are exposed to contamination, the most contaminated and perhaps only contaminated items may be the soldiers' footwear. Prior to entering vehicles, aircraft, etc. soldiers should decontaminate. Individual decontamination of footwear in a location where a soldier cannot sit and where he or she cannot remove footwear would be most difficult. This invention provides a decontamination device that allows soldiers to decontaminate footwear without removing them and without sitting down. This invention provides soldiers with something they presently do not have—a portable, disposable, footwear decontamination device they can set up next to an open door, then step directly from the device into a vehicle or aircraft.

### Decontamination Stations

Decontamination stations rely primarily on the method of decontaminating or washing outer surfaces, systematic removal of contaminated clothing, and showering each individual. Decontamination planning includes a “shuffle pit” or “shuffle box” at the starting point of a decontamination station. A shuffle pit is an area, often a shallow ditch, where soldiers remove gross contamination from their boots. Most often, the shuffle pit/box contains sand and/or a dry decontaminating formulation. A commercially-produced 3 ft. by 5 ft. shuffle box is available in the military inventory as part of a decontamination station kit (National Stock No. 8145-01-522-2526 and National Stock No. 8145-01-527-1020), and is described in U.S. Pat. No. 7,299,811. The commercial shuffle box is a large, shallow box with straight sides, and is capable of holding dry or liquid decontaminating formulations.

### Reactive Components

Decontamination of hazardous materials may be accomplished in a number of ways. A decontaminating formulation may react with a hazardous material as an oxidizer, hydrolizer, neucleophile, electrophile, or perhaps as a poly-reactive formulation. Decontamination of both chemicals and pathogenic microorganisms involve chemical reactions. The most common methods used to decontaminate hazardous materials are chlorinating oxidation and alkali hydrolyzation.

The type of chemicals used most often as reactive components in decontaminating formulations are those used in homes and industries as bleaching agents. Bleaching agents can be categorized as halogen donors and peroxygen compounds. Halogen donors include compounds such as sodium hypochlorite, calcium hypochlorite, sodium hydroxide, chlorinated lime, and sodium dichloro-s-triazinetrione. Examples of peroxygen compounds include hydrogen peroxide, sodium percarbonate, peracetic acid, calcium peroxide, and sodium borate. A wide variety of bleaching agents may be used in the decontamination side of this invention. See U.S. Pat. No. 5,612,305, which is hereby incorporated by reference for additional examples of bleaching agents.

### Adjuvants

Decontaminating formulations are often a mixture of one or more chemically-reactive compounds and one or more adjuvants. An adjuvant may be a reactant, substrate, reagent, or a component that does not react with the target residue. An adjuvant contributes to the chemical reaction that results in decontamination, but without a reactive component, cannot detoxify or neutralize the target residue. Some adjuvants are called by a name that describes one of its contributions to a reaction. An adjuvant may be referred to as a surfactant, wetting agent, spreader, sticker, extender, penetrant, compatibility agent, doping agent, buffer, pH modifier, foaming agent, defoamer, and solvent.

Common adjuvants are the alkyl benzene sulfonates, branched-chain anionic surfactants, linear alkyl benzene sulfonates, straight-chain anionic surfactants, alkyl phenoxy polyethoxy ethanols, octyl phenoxy polyethoxy ethanols, alkyl ammonium chlorides, alkyl glucosides, and detergency builder materials. Detergency builder materials have the ability to remove metal ions other than alkali metal ions, and increase alkalinity. Examples of detergency builders include the sodium phosphates, sodium silicates, sodium carbonates, and sodium borates.

### Deactivating Formulations

Decontaminating formulations detoxify and neutralize hazardous materials, but they can damage leather and rubber, bleach color from carpets, corrode metal and other key materials, and deposit unwanted residues on floors, equipment, and vehicles. In the existing art, there are no instructions or provisions for deactivating decontaminating formulations remaining on footwear. Decontamination doctrine for the U.S. Armed Forces, such as FM 3-5 and FM 3-11, includes instructions to decontaminate boots, and when feasible—rinse, but no reference or instruction regarding the deactivation of decontaminating solutions from footwear is found. This invention proposes an improvement to military decontamination operations.

Like decontaminating formulations, deactivating formulations often include an “active ingredient” and an adjuvant. Deactivating formulations can be used to adjust pH and to bind elements in the decontaminating formulation. Two common pH adjusters are sodium dihydrogen phosphate for bases and sodium carbonate (bicarbonate) for acids. Elements or formulations that bind elements to change their properties are sometimes referred to as “doping agents.” Doping agents are used in decontaminating formulations and deactivating formulations. Where a halogen such as chlorine is a major component of the decontaminating formulation, a doping agent such as sulfur is often used. Examples of active ingredient compounds for deactivating formulations include sodium sulfite, sodium thiosulfate, sodium pyrophosphate, sodium polyphosphate, sodium borate, and ethylene diamine tetra-acetic acid (EDTA).

### NATURE OF THIS INVENTION

In the preferred embodiment, this invention provides a portable, disposable device and method for decontaminating footwear while the footwear are being worn. The device of the preferred embodiment consists of two basins attached together on one side with a hinge. The general appearance is that of a suitcase with the top and bottom halves equal in size. The device is opened for use and placed on the ground or floor near a contaminated area. One basin will be closer to the contaminated area than the other.

A chemically-reactive, neutralizing formulation (decontaminating formulation) is placed in the basin closest to the contaminated area and a chemically-reactive rinsing formulation (deactivating formulation) is placed in the other basin. Persons using a device of this invention may use the decontaminating and deactivating formulations provided with the device, or use formulations they provide if better suited for their needs. Where decontaminating chemicals or deactivating chemicals must be mixed prior to use, mixing may be done in the basin where the formulation will be used.

A person wearing contaminated footwear steps into the first basin (the decontaminating side) and moves their feet in a stepping or sliding motion to remove or decontaminate residues. Hazardous materials are either decontaminated on the footwear or removed from footwear then decontaminated in the basin. The person then steps into the second basin (the deactivating side) and moves their feet in a stepping or sliding motion. A chemical reaction between traces of the decontaminating formulation and the deactivating formulation changes the properties of the decontaminating formulation making it less damaging.

The person could then step onto a towel (provided) or other absorbent material to remove liquid from their footwear. Drying footwear right after decontamination may not be feasible or needed in every situation. For convenience, where deactivation of the decontaminating formulation is not necessary, the second basin may be used for additional decontaminating formulation or for a place to step on the towel.

The device of this invention may be used alone or placed end-to-end or side-by-side with one or more additional devices. When finished decontaminating with this device, the user places absorbent material (provided) into the liquid decontaminating and deactivating formulations. The absorbent material expands into a gel-like substance as it absorbs the liquids. After the gel is formed, the user closes the device and closes the latches to keep the device from opening unexpectedly.

The device of this invention is designed to be leak-resistant but sealing tape (provided) may be applied to the seam as an added safety measure. The user then attaches hazardous material warning labels (provided) to both sides of the device. The warning labels state that hazardous materials may be inside the device and the device should not be opened. Where safety dictates, the device may be placed inside a labeled and sealable polyethylene sleeve (plastic zip-close bag). Disposing of a device of this invention should always comply with applicable laws and the organization's procedures.

The following examples are not inclusive of all situations and uses but are included to assist in visualizing how this invention may be used. At accident scenes, emergency medical technicians often walk through blood and other body fluids. EMT's can use this invention to decontaminate their footwear for bloodborne pathogens and other hazards. Firefighters respond to accident scenes, chemical spills, and other emergencies where they may walk through hazardous materials. Firefighters also provide decontamination to other government officials and the public at hazardous situations.

Firefighters can use this invention to decontaminate their own footwear and the footwear of other government officials and the public. This invention works well as the first treatment point in a decontamination station 8). Police officials serve arrest warrants at hazardous locations such as clandestine drug labs. Officers can use the device of this invention to decontaminate their footwear as they leave a clandestine drug lab to prevent transferring hazardous chemicals to the floor of their police vehicles, then later to their homes.

At a crime scene, such as a homicide, after the crime scene is secured, police officials may deploy two of the devices of this invention and use one prior to entering and the other prior to leaving (6). Using the device prior to entering can preclude contaminating the scene with matter brought from other locations on the officers' and investigators' shoes. Prior to leaving the area, the second device can be used to decontaminate footwear and prevent spreading contaminants to public areas, vehicles, and other crime scenes. Corrections employees in prisons and jails can use this invention in the same way.

In a military environment, soldiers can use this invention to decontaminate their boots prior to entering a vehicle or aircraft to be transported to a decontamination station. This device can be used on naval vessels, with armored vehicles (tanks) and trucks, and in defensive emplacements and garrison areas where a spray from aircraft or exploding munitions might have contaminated exterior surfaces but where contamination is not yet confirmed. Prior to passing from the outdoor area to the interior, personnel can decontaminate their footwear as a precaution.

In a commercial environment, this invention can be used where workers walk from different areas of a building such as from the meat-packing area or manufacturing area, into the front office. Decontamination workers who decontaminate drug labs, sewage, mold, hantavirus, mercury, lead, and other hazardous sites can use this invention as they pass from one area to another and as part of a decontamination station.

Differences from Existing Art

Some of the significant differences between this invention and the existing art are as follows:
1. This invention uses a deactivating solution to deactivate the decontaminating formulation.
2. This invention provides for a side configuration that reduces the possibility of liquids splashing over a side (7).
3. This invention provides for its use at locations where toxic chemical residues may be present.
4. This invention provides for its use at locations such as crime scenes and accident scenes where bloodborne pathogens and other body fluid hazards may be present.
5. This invention provides for its use at locations where radiological residue may be present.
6. This invention provides for its use at locations where body fluids that may contain DNA or other properties that may contaminate a crime scene may be present.
7. This invention provides for the use of multiple devices for faster decontamination of many people or repeated decontaminating measures.
8. This invention provides the means for absorbing liquids within the device, enclosing the device, then sealing the device, all to prevent liquids from leaking after the device is used.
9. This invention provides labels to mark a used device as a container of potentially hazardous materials.
10. This invention provides for the use of dry decontaminating formulations.

### BRIEF DESCRIPTION OF THE DRAWINGS

FIG. 1 represents a generalized conceptualization of the preferred embodiment of this invention.

FIG. 2 represents a generalized top view of a square embodiment of this invention.

FIG. 3 represents a side view of the upper-most edge of two basins that fit together in a “tongue-and-groove” configuration to form a water-tight seal.

FIG. 4 represents a side view of a square or rectangular embodiment of this invention that illustrates a pad, mat, grate, mesh, or other material or surface on the bottom, inside each basin.

FIG. 5 represents a generalized top view that illustrates two devices placed end-to-end.

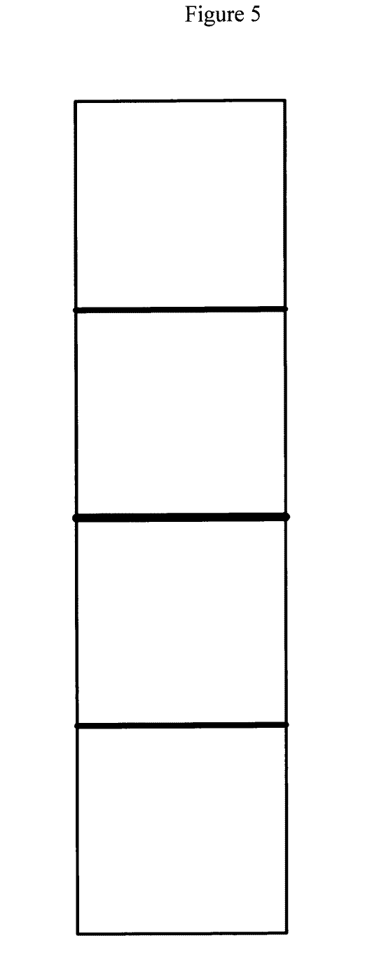

FIG. 6 represents a generalized top view that illustrates two devices placed side-by-side.

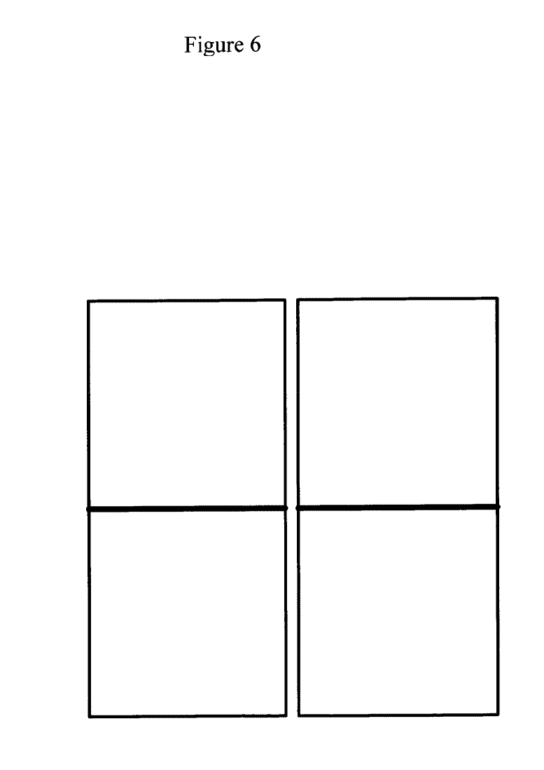

FIG. 7 represents side views of possible side embodiments for this invention.

FIG. 8 represents a decontamination station that depicts this invention as the first component of the decontamination station.

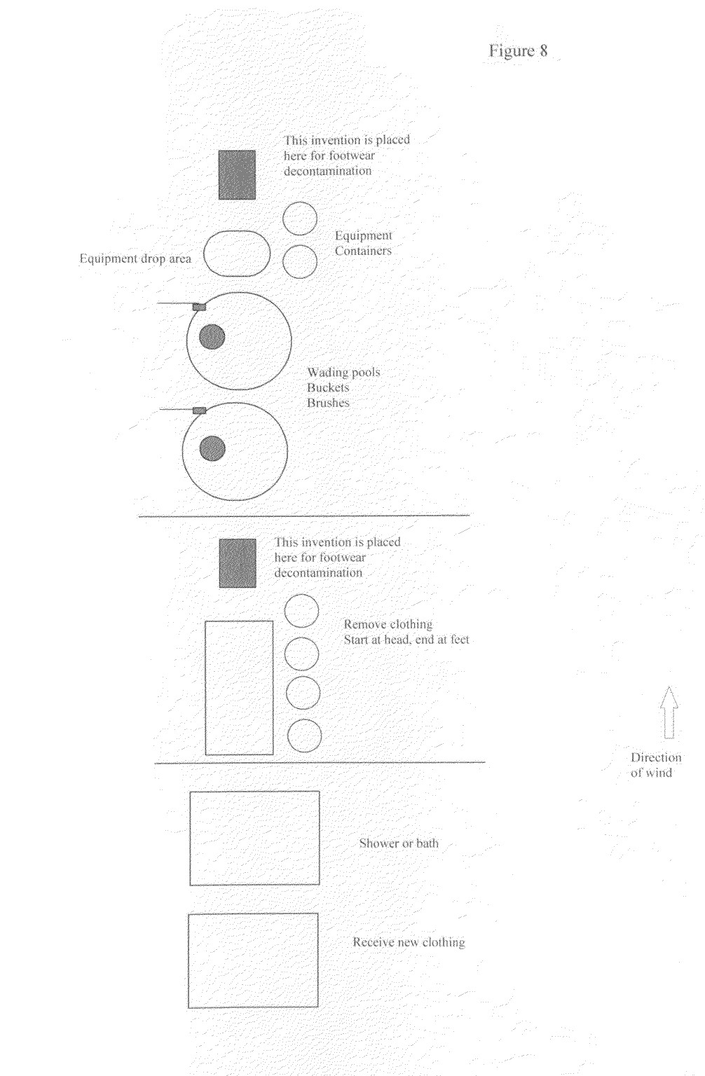

FIG. 9 represents a generalized conceptualization of this invention that illustrates the device placed by a doorway.

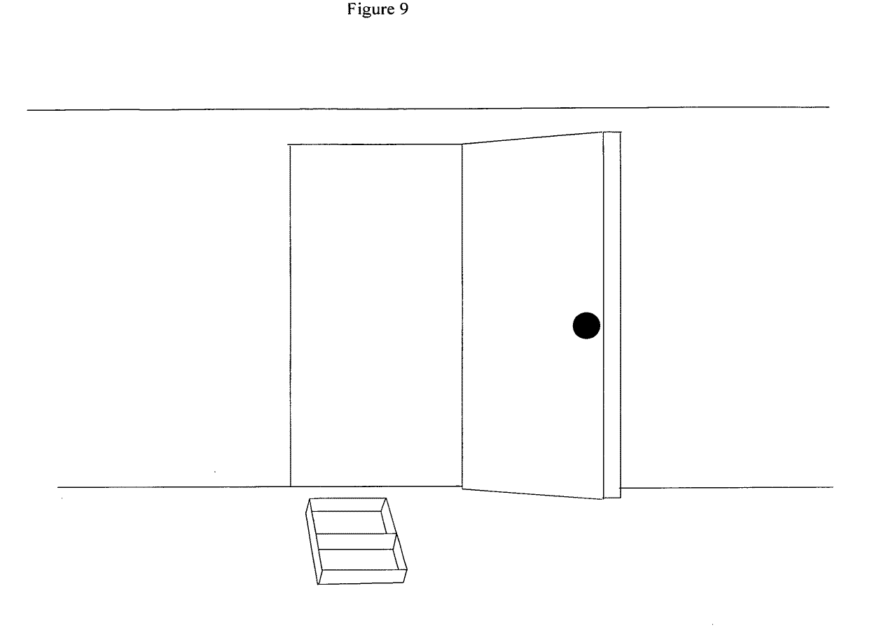

FIG. 10 represents a generalized conceptualization of this invention that illustrates the placement of warning labels on exterior surfaces.

Note: These drawings are provided to illustrate different characteristics of this invention. Details were usually excluded from drawings where including the missing details would not contribute to the purpose of the drawings. These drawings are not intended to illustrate all details and embodiments of this invention.

We claim:

1. A decontaminating device and method that decontaminates residue on and from footwear and other surfaces, configured as two basins, with a decontaminating formulation in one basin and a deactivating formulation in the other basin.

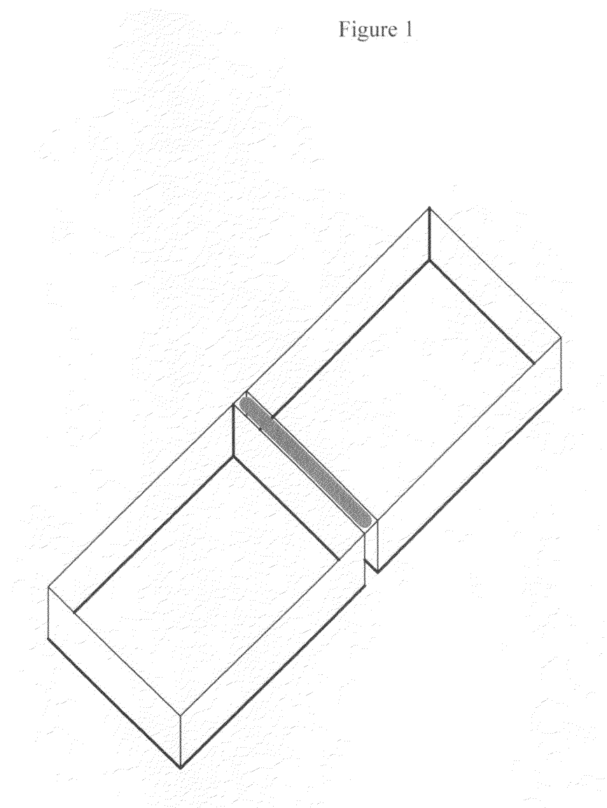

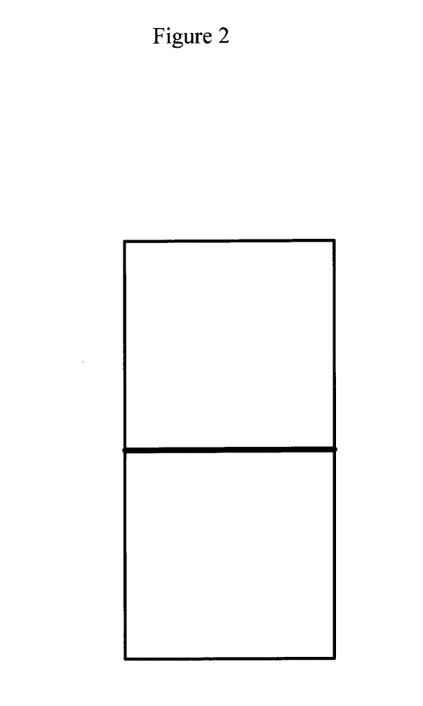

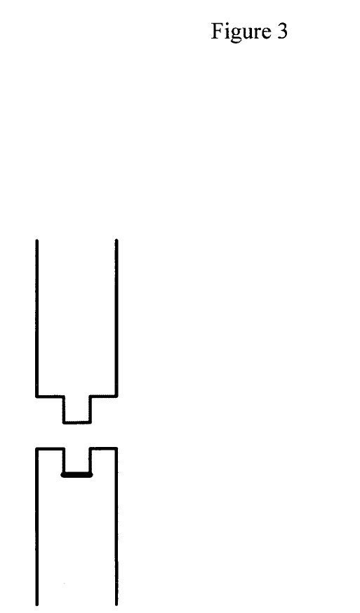

2. The device and method of claim 1 comprising two, square basins, each 6 inches high, 20 inches wide, and 20 inches long; open at the top, with both openings facing skyward; each basin enclosed on the bottom and all sides, capable of retaining a liquid without the liquid leaking through a side or bottom (FIG. 1, FIG. 2); attached at a mutual side with at least one hinge, capable of being closed suitcase-like where the inside of both basins are enclosed, and with at least one latch appurtenance capable of causing both basins to be pressed and held together along the edges of their openings; where the upper-most edge at the opening of one basin has a groove configuration and the upper-most edge at the opening of the other basin has a lip configuration that fits within the groove of the first basin and contacts a pliable material to form a seal-like union resistant to the passage of liquids through the union (FIG. 3).

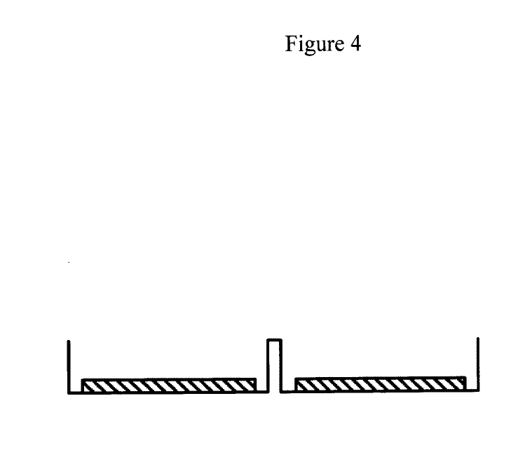

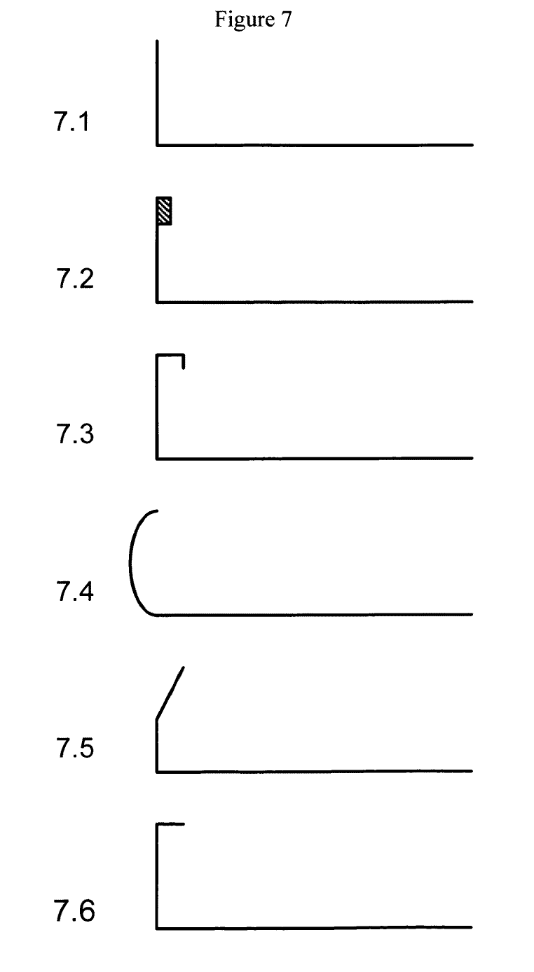

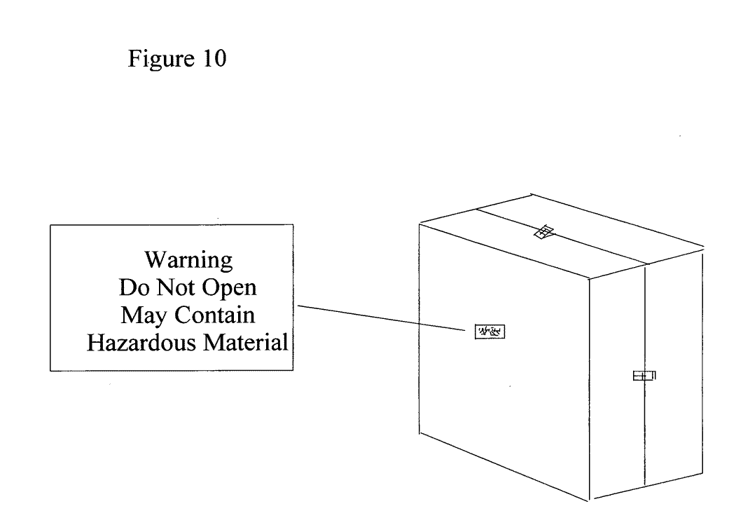

3. The device and method of claim 1 and 2 comprising two basins, where inside each basin, near the opening, material protrudes toward the center of each basin to reduce the ease at which liquid may splash from each basin (FIG. 7.2); and comprising one abrasive pad each, situated or affixed within each basin to provide friction to the bottom or other exterior surfaces of footwear (FIG. 4), which may depress and release some of the liquid from within when pressed from above by footwear; and comprising one or more rough, textured surfaces or materials on the area of each basin that contacts the floor or ground when the device is in use, that may reduce the possibility of the device sliding or inadvertently moving while being used (FIG. 10).

4. The device and method of claim 1 comprising one or more chemically-reactive elements, compounds, or mixtures as a liquid, decontaminating formulation placed in the decontamination basin of the device and capable of decontaminating at least one hazardous element, compound, mixture, radioactive species, pathogenic microorganism, or microbial toxin, or capable of changing a genetic or chemical property of at least one body fluid, which may include blood, blood derivative, urine, saliva, bile, mucus, feces, or other liquid or viscus matter produced by a person or animal; and comprising one or more chemically-reactive elements, compounds, or mixtures as part of a liquid, deactivating formulation placed in the deactivation basin of the device, and capable of reducing a reactive property or other undesired property of the decontaminating formulation used at that time in the other basin of the device; where a person wearing contaminated footwear steps into the first basin (the decontaminating side) and moves their feet in a stepping or sliding motion to remove or decontaminate residues, then the person steps into the second basin (deactivating side) and moves their feet in a stepping or sliding motion to deactivate the decontaminating solution residue that is on their footwear, then, if feasible and necessary, the person may step from the deactivating basin onto a towel or other absorbent material to remove excess liquids from their footwear.

5. The device and method of claim 1 comprising at least 4 ounces, by volume, of liquid-absorbing sodium polyacrylate, at least 10 linear feet of sealing tape that is capable of remaining firmly attached to the basin surfaces of the device when exposed to moisture, and at least two warning labels (FIG. 12); where the sodium polyacrylate is placed in the liquids in the basins after the user is finished using the device but prior to discarding the device; where after the sodium polyacrylate absorbs all liquid or a substantial majority of the liquid in the basins, the user then closes the device by folding at the hinge and placing the openings together; where the user then operates all latches and applies sealing tape over the seam created where the tops of the basins meet; where the user then applies one warning label near the center of the outside bottom of each basin; and where the user places the device inside a self-sealing, impervious envelope.

6. An embodiment of the device and method of claim 1 comprising two basins, each open at the top, enclosed on the bottom and all sides with the openings facing skyward in the same direction; and capable of retaining a liquid without the liquid leaking through a side or bottom(1); attached at a mutual side with at least one hinge or other attachment means, capable of being closed suitcase-like where the inside of both basins are enclosed, and with at least one latch appurtenance, elastic-like band, or other means capable of firmly pressing and holding the openings of both basins together; where one basin has a gasket-like material that forms a seal-like union resistant to the passage of liquids through the union when the upper-most edges of both basins are pressed or held together;
a where one or more basins are no less than 1 inch but no more than 24 inches deep;
b. where one or more basins are 12 inches to 62 inches wide and 12 inches to 62 inches long and large enough in width and length to permit a person's foot and footwear to be placed inside on or near the bottom without the foot or footwear contacting any side;
c. where one or more basins have a design generally recognizable as square, rectangular, round, triangular, or other geometrical-like design; and have the general shape of a basin, tray, vessel, pan, bucket, or tub(2);
d. where moisture-resistant tape with an impervious backing is placed over opening edge where basins attach to preclude liquid from passing through the attachment point.

7. The device and method of claim 6 comprising one or more chemically-reactive elements, compounds, or mixtures placed in the decontamination basin of the device and capable of decontaminating at least one hazardous element, compound, mixture, radioactive species, pathogenic microorganism, or microbial toxin, or capable of changing a genetic or chemical property of at least one body fluid or other liquid or viscus matter produced by a person or animal; and comprising one or more chemically-reactive elements, compounds, or mixtures as part of a liquid, deactivating formulation placed in the deactivation basin of the device, and capable of reducing a reactive property or other undesired property of the decontaminating formulation used at that time in the other basin of the device; where a person wearing contaminated footwear steps into the first basin (the decontaminating side) and moves their feet in a stepping or sliding motion to remove or decontaminate residues, then the person steps into the second basin (deactivating side) and moves their feet in a stepping or sliding motion to deactivate the decontaminating solution residue that is on their footwear;
a. where the device and method of claim 6 comprises a halogen-donating compound, peroxygen compound, oxidizing compound, hydrolyzing compound, neucleophilic compound, electrophilic compound, bleaching agent, commercially-produced decontaminating formulation, or pesticide as defined by the Federal Insecticide Fungicide and Rodenticide Act (FIFRA) as the chemically-reactive component in the decontaminating formulation placed in the basin of the device of this invention in a liquid concentration between 0.2% and 100% by volume; or as a dry formulation with a concentration between 0.5% and 100% by volume;
b. where the device and method of claim 6 comprises a decontaminating formulation comprised of a chemically-reactive element, compound, or mixture, that need not be mixed with an adjuvant, but when mixed with an adjuvant in a aqueous solution, the chemical components of the adjuvant other than water are concentrated between 0.1% and 10% by volume; or when mixed with an adjuvant in an anhydrous formulation, the adjuvant has a concentration between 0.5 and 99.5% by volume;
c. where the device and method of claim 6 comprises one or more pH adjusting elements, compounds, or mixtures, or comprises one or more binding or doping elements, compounds, or mixtures; and may be comprised of both a pH adjusting component and binding or doping component;
d. where the device and method of claim 6 comprises a sufficient depth of decontaminating formulation or sufficient depth of deactivating formulation to contact surfaces of footwear above the sole.

8. The device and method of claim 6 comprising two basins where the side material of each basin has a bend toward the inside of the basin at a ninety degree angle from the side, which extends horizontally toward the inside of each basin all the way around for about one inch, where the continued side material of both basins bends ninety degrees again and extends about one inch toward the inside bottom of each basin (FIG. 7.1) or where the upper-most edge of the opening of one or more basins have one or more angled, coped, or curved sides which are angled, coped or curved toward the inside of the basin in such a manner that may inhibit or preclude liquid inside from easily spilling over the side (FIG. 7), or comprising one or more sides of the basin with a lip, extension, or angled edge at or near the top and situated on the inside surface of the side in such a manner that may inhibit or preclude liquid from easily spilling over the side (FIG. 7).

9. The device and method of claim 6 comprising one or more anchors or anchor points of attachment that allows one or more basins to be anchored to the floor, ground, wall, or other lower surface to inhibit or prevent a basin or device from sliding or otherwise moving during use.

10. The device and method of claim 6 comprising at least one abrasive pad, mat, grate, mesh, brush, or other material or surface placed, situated, or affixed on or within at least one basin to provide friction to the bottom or other exterior surface of footwear or to move decontaminating formulation or deactivating formulation from the basin to an exterior surface of footwear.

11. The device and method of claim 6 comprising 4 or more ounces of one or more liquid-absorbing materials such as absorbent polymer, silica gel, colloidal silica, activated alumina, soda lime absorbent, clay hydro-gel, polyacrylamide copolymer, ethylene maleic anhydride copolymer, cross-linked carboxy-methyl-cellulose, polyvinyl alcohol copolymers, cross-linked polyethylene oxide, and starch grafted copolymer of polyacrylonitrile, or other absorbent; provided with the device of this invention, and subsequently placed in any liquid inside the device after the device is used but prior to discarding the device; where the absorbent matter or material absorbs all liquid or a substantial majority of the liquid.

12. An embodiment of the device and method of claim 1 comprising two basins provided together in a kit where the basins are not attached to each other but otherwise have the same attributes, configurations, materials, methods, and utility as the embodiment described in claim 6.
